(19) United States
(12) Patent Application Publication
(10) Pub. No.: US 2013/0085353 A1
(43) Pub. Date: Apr. 4, 2013

(54) OPTICAL BACKSCATTERING DIAGNOSTICS

(71) Applicants: Mario Ettore Giardini, North Haugh (GB); Thomas Fraser Krauss, North Haugh (GB); Andrea Di Falco, North Haugh (GB)

(72) Inventors: Mario Ettore Giardini, North Haugh (GB); Thomas Fraser Krauss, North Haugh (GB); Andrea Di Falco, North Haugh (GB)

(73) Assignee: UNIVERSITY COURT OF THE UNIVERSITY OF ST ANDREWS, St Andrews (GB)

(21) Appl. No.: 13/639,609

(22) PCT Filed: Apr. 6, 2011
(86) PCT No.: PCT/GB11/00527

§ 371 Date: Dec. 12, 2012

(30) Foreign Application Priority Data
Apr. 9, 2010 (GB) 1005919.4

(51) Int. Cl. A61B 5/1455; A61B 5/00

(52) U.S. Cl. CPC A61B 5/14552; A61B 5/14556; A61B 5/7228
USPC 600/317; 600/323

(57) ABSTRACT

A system for non-invasive measurement of parameters relating to a biological tissue comprising: a plurality of light sources, each operable to emit a light signal with one or more predetermined wavelengths; a light detector for detecting light from the tissue as a result of illumination by the sources, and means for applying modulation functions to the light emitted to implement code division multiplexing, each source being associated with a different function, wherein the functions are selected so that there is substantially no cross correlation between them, and wherein the sources have overlapping spectra.

### FIELD OF THE INVENTION

The present invention relates to a system and method for monitoring light transmission and/or backscattering.

### BACKGROUND OF THE INVENTION

Light transmission and/or backscattering, typically in the near-infrared, is a well known technique for monitoring blood and other biological tissue constituents. It allows, for example, the degree of oxygenation of such tissues to be established. This is because haemoglobin and myoglobin have different near-infrared optical absorption spectrum depending on whether they are in an oxygenated or deoxygenated state. The oxygenation state can be determined by shining light on the tissue and observing the transmitted or backscattered light intensity. As another example, the content of cytochrome aa₃ oxydase in tissue can be determined in a similar way.

Monitoring oxygenation levels is very useful, for example during surgery, as tissue needs to be interrogated in order to establish whether it is correctly perfused by blood. Other applications include emergency care medicine, for the determination of the oxygenation state of brain tissue; sports medicine and rehabilitative cardiology, for the determination of the oxygenation state of muscle haemodynamics and of capillary contractility; vascular surgery, for the determination of blood vessel elasticity by observation of the response of vascularised tissue to adequate stimuli; catheterised tools, as a navigation aid via the identification of different types of tissues through their optical backscattering and/or transmission properties.

U.S. Pat. No. 5,807,261 describes a tool for non destructive interrogation of tissue. This has a light source and light detector, which may be mounted directly on the tool or mounted remotely and guided to the surgical field using fibre optic cables. The light source used may be broadband. In this case wavelength differentiation can be done at the detector using filters or gratings or using time, frequency or space resolved methods. Alternatively, discrete monochromatic sources may be provided which are subsequently multiplexed into a single detector using time or frequency multiplexing. Whilst the tool of U.S. Pat. No. 5,807,261 has advantages, a problem is that it relies on narrowband optical filtering, which is complex to attain and therefore requires additional components. Also, where multiple sources are used, the frequency domain and time domain multiplexing proposed are both sensitive to narrowband and spiking noise.

WO03/014714 describes a spectrometer that uses spread spectrum multiplexing techniques. This has better rejection to narrowband electromagnetic interference than frequency domain and time domain multiplexing. However, spread spectrum multiplexing suffers from source crosstalk due to the small cross-correlation of the known spread-spectrum modulation codes.

### SUMMARY OF THE INVENTION

According to a first aspect of the invention, there is provided a system for non-invasive measurement of parameters relating to a biological tissue comprising: a plurality of light sources, each operable to emit a light signal with one or more predetermined wavelengths; a light detector for detecting light from the tissue as a result of illumination by the sources, and means for applying modulation functions to the light emitted to implement code division multiplexing, each source being associated with a different function, wherein the functions are selected so that there is substantially no cross correlation between them, and wherein the sources have overlapping spectra.

Demultiplexing means may be provided for determining the contribution of each of the sources to the signal detected by the detector.

The modulation means may use Hadamard or reduced-Hadamard (Simplex) code sequences.

The parameter may be the oxygenation of the biological tissue.

Light from the tissue may be one or more of transmitted light; reflected light; backscattered light; fluorescence spectroscopy, Raman emission.

Light from at least one and preferably both of the sources may be in the infrared region.

Each source may have a wavelength bandwidth in the range 10-140 nm. For example, each source may have a wavelength bandwidth in the range 20-100 nm. Each source may have a wavelength bandwidth of more than 100 nm.

According to another aspect of the present invention, there is provided a tool that includes a system according to the first aspect of the invention, the tool comprising a proximal or handle portion that includes the plurality of light sources and the detector, and a distal portion, wherein at least one optical fibre extends between each light source and the distal end for allowing the transmission of light to the distal end for transmission into tissue, and at least one optical fibre extends from the detector to the distal end to capture light that has passed through the tissue.

The tool may have gripping portions for gripping tissue. The plurality of light sources and the detector may be positioned on one or more gripping faces of the gripping portions.

### BRIEF DESCRIPTION OF THE DRAWINGS

Various aspects of the invention will now be described by way of example only and with reference to the accompanying drawings, of which:

FIG. 1 is a schematic diagram of a backscattering measurement tool for measuring optical characteristics of tissue;

FIG. 2 shows spectra of two sources used in the tool of FIG. 1, and

FIG. 3 is schematic diagram of a transmission measurement tool for measuring optical characteristics of tissue.

### DETAILED DESCRIPTION OF THE DRAWINGS

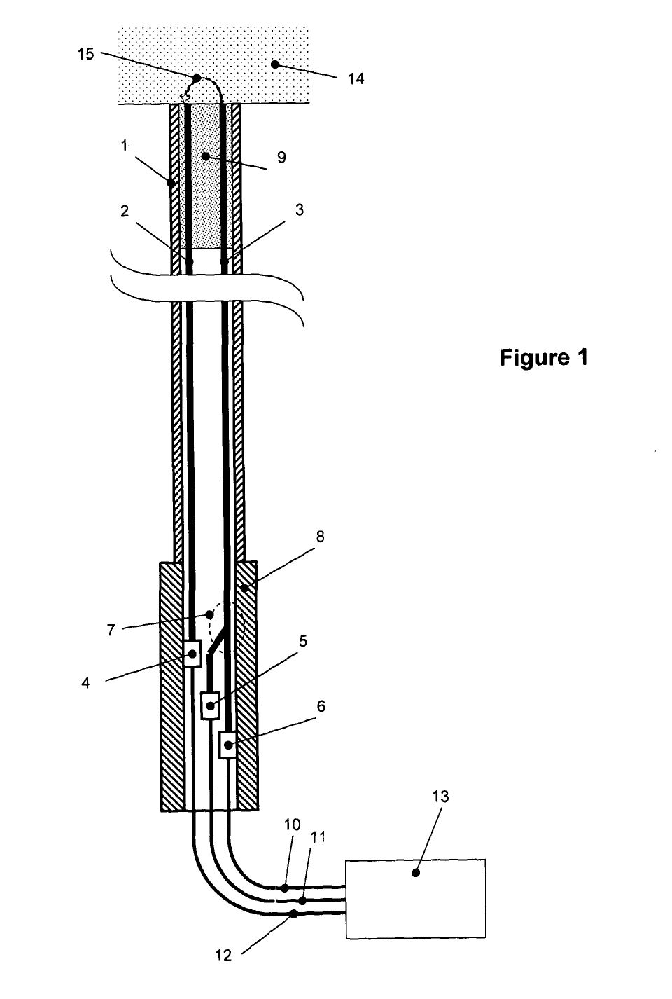

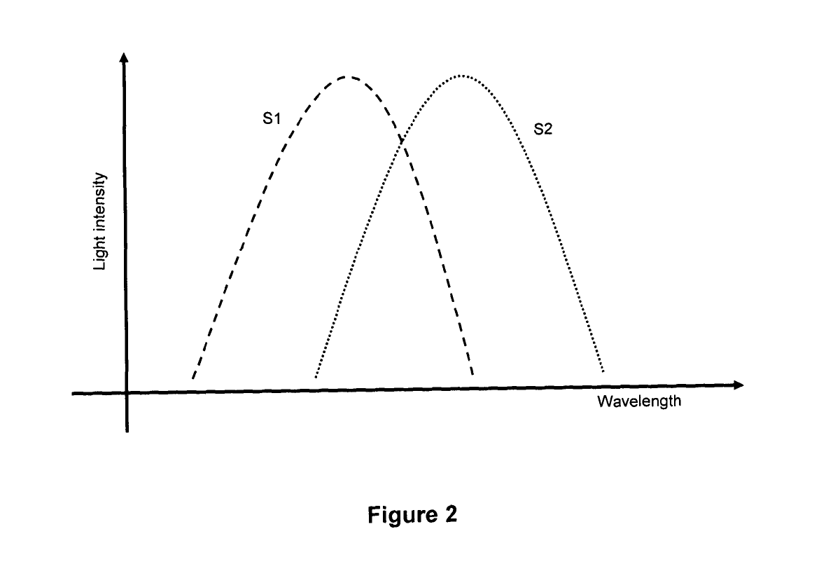

FIG. 1 shows a backscattering measurement tool for interrogating tissue for oxygenated and deoxygenated haemoglobin content. This has two broadband sources S1 and S2, for example unfiltered light emitting diodes (LEDs), at near-infrared wavelengths λ₁ and λ₂ with spectra S1(λ) and S2(λ), as shown in FIG. 2. The tool has a metal tube 1 of about 5 mm diameter and 40 cm length. In the tube are two optical fibres 2 and 3. Fibre 2 is connected to a photodiode 4, while fibre 3 is connected to two infrared LEDs 5 and 6 by means of a Y splitter 7. The LEDs and photodiodes are connected using cables 10, 11 and 12 to external driver and detection electronics 13. The electronics provided are arranged to modulate the input light in such a manner as to implement code division multiplexing. This will be described in more detail later.

The sources and detectors are contained in a handle 8 at the proximal end of the tube. At the distal end, the tube is filled with a potting compound 9. This ensures optical separation between the fibres, and seals the distal end to liquids and gases. The distal end of the tool can be sterilised, e.g. by hot steam or gases, and used on surgically exposed tissue, both in open field and in laparoscopy. For the latter use, it may be advantageous for simpler access to the tissue to cut the distal end at an angle, e.g. of 5-30 degrees, and/or to use a curved and/or flexible tube/distal end.

In use, the tool is positioned so that it touches the tissue to be investigated 14 with the distal tip. The two LEDs 5 and 6 emit light 15 through the fibre 3 into the tissue. Backscattered light is collected by fibre 2 and conveyed to the photodiode 4. The electronics identifies how much light coming from each source is backscattered, and applies the algorithms necessary to extract the relevant data.

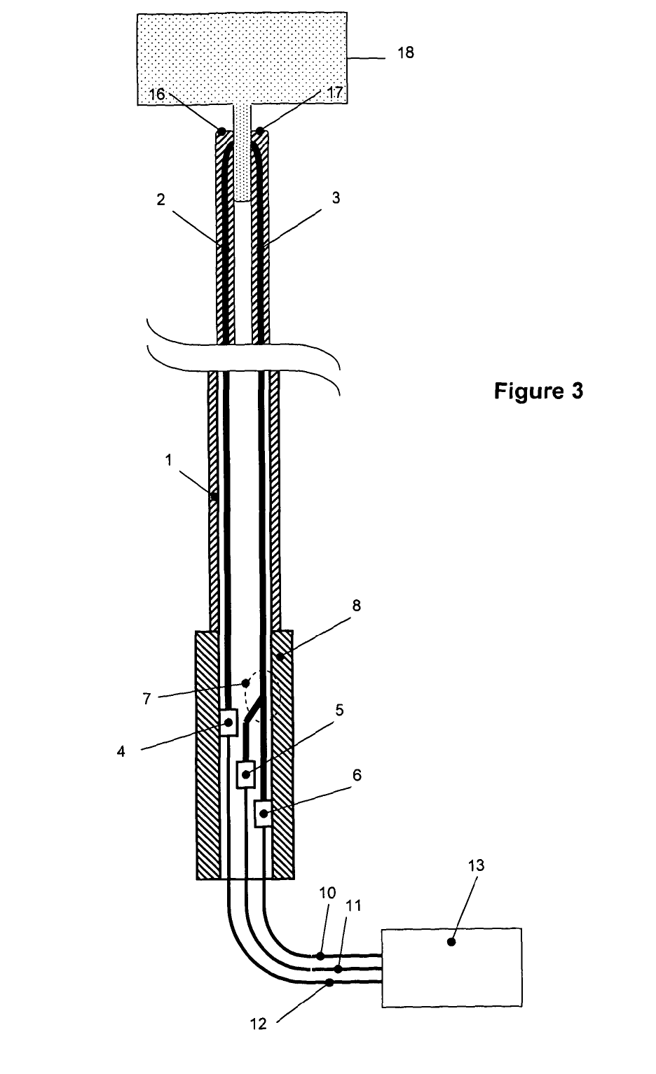

FIG. 3 shows a transmission measurement tool coupled to the tip of a surgical grasping tool. Again, a metal tube 1 of about 5 mm diameter and 40 cm length contains two optical fibres 2 and 3. Fibre 2 is connected to a photodiode 4, while fibre 3 is connected to two infrared LEDs 5 and 6 by means of a Y splitter 7. As before, the LEDs emit at near-infrared wavelengths λ₁ and λ₂ and have spectra S1(λ) and S2(λ), as shown in FIG. 2. Cables 10, 11 and 12 connect the LEDs and photodiodes to external driver and detection electronics 13. The electronics provided are arranged to modulate the input light in such a manner as to implement code division multiplexing and analyse the light received at the detector to extract the relevant data.

On the photodiode/LED end of the tube (proximal end), sources and detectors are contained in a handle 8. On the other end (distal end), the tube presents two graspers 16 and 17, that can be closed and opened with a pincer-like movement to grasp tissue.

In use, the tissue to be investigated 18 is grasped with the distal tip graspers 16 and 17. The two LEDs 5 and 6 emit light through the fibre 3 into the tissue. Transmitted light is collected by fibre 2 and conveyed to the photodiode 4.

The devices of FIGS. 1 and 2 can be used to interrogate tissue for oxygenated and deoxygenated haemoglobin content. This can be done using well known algorithms for narrowband sources, but adapted to take into account the broadband nature of the spectra. The known algorithms use absorption coefficients for oxygenated haemoglobin and deoxygenated haemoglobin. For the present invention, these have to be adapted to take into account the wavelength variation over the relatively wide bandwidth of the sources. The adapted algorithms may be as follows:

α₁^{HbO2}=∫S1(λ)α^{HbO2}(λ)dλ

α₂^{HbO2}=∫S2(λ)α^{HbO2}(λ)dλ

α₁^{Hb}=∫S1(λ)α^{Hb}(λ)dλ

α₂^{Hb}=∫S2(λ)α^{Hb}(λ)dλ

where λ is the wavelength; S1(λ) and S2(λ) are the spectra for the sources; α^{HbO2}(λ) is the absorption coefficient spectrum for oxygenated haemoglobin; α^{Hb}(λ) is the absorption coefficient spectrum for deoxygenated haemoglobin; and α₁^{HbO2}, α₁^{Hb}, α₂^{HbO2}, α₂^{Hb} are used as equivalent absorption coefficients for sources S1 and S2 in the algorithms known in the literature. The integrals are calculated over the full emission bandwidth of each source. As exemplified in FIG. 2, the source spectra may overlap, as long as the equivalent absorption coefficients are different from each other. Such overlap is admissible when the equivalent absorption coefficients, calculated from the preceding formulas, differ from each other by at least 1%, and preferably by more than 5%.

In the tools described above, the signal S detected by the photodiode 4 can be written as:

$$S = {\sum }_{n = 1 … N} {a}_{n} ( t ) {f}_{n} ( t ) dt$$

where N is the number of sources (in this case, 2) and fₙ are N functions used to modulate the source intensity. To avoid crosstalk between the sources, the modulation functions are selected to have substantially no cross-correlation, i.e.

∫fₙ(t)fₘ(t)dt=0 when n≠m

where t is time, the functions are periodic sequences of the values −1 and 1 with the same period, and the integral is calculated over the period. Also, fₙ may be functions differing by phase or delay only, as long as the condition remains valid.

To satisfy the above condition, code-division multiplexing (CDM) is used. Any CDM sequence satisfying the vanishing cross-correlation condition can be used. As an example Hadamard or reduced-Hadamard (Simplex) code sequences are employed. Such sequences are described in M. Harwit and N. Sloan, Hadamard Transform Optics (1979, Academic Press, U.S.A.). The Hadamard sequences give an optimised signal-to-noise ratio. The Simplex sequences are not quite as optimal but are simpler to generate with linear shift generators.

Using the vanishing cross-correlations, the signal component Sₙ corresponding to source n can be calculated and measured as

Sₙ=∫S(t)fₙ(t)dt

Techniques can be employed to compensate for the fact that the functions fₙ oscillate between 1 and −1, while light intensity can oscillate between positive or null values only. Techniques for doing this are well known and so will not be described in detail.

Once the signal is demodulated, the components can be analysed to determine any parameters of interest, for example the oxygenation levels. As noted above, techniques for doing this are known and so will not be described in detail.

The invention can be advantageously extended to any optical spectroscopy technique that can benefit from the application of one or more sources to biological tissue, and from the assignment, on one or more detectors, of the signal contribution deriving from each source. For example, the invention could be applied to transmission and/or backscattering spectroscopy, fluorescence spectroscopy, Raman scattering.

A skilled person will appreciate that variations of the disclosed arrangements are possible without departing from the invention. Accordingly the above description of the specific embodiment is made by way of example only and not for the purposes of limitation. It will be clear to the skilled person that minor modifications may be made without significant changes to the operation described.

The work leading to this invention has received funding from the Commission of the European Communities Information Society and Media Directorate—General Information and Communication Technologies—Seventh Framework Programme, a Collaborative Project entitled “Array of Robots Augmenting the KiNematics of Endoluminal Surgery” (ARAKNES) ([FP7/2007-2013) under grant agreement no 224565.

1. A system for non-invasive measurement of parameters relating to a biological tissue, the system comprising:
a plurality of light sources, each operable to emit a light signal with one or more predetermined wavelengths; and
a light detector for detecting light signals from the biological tissue as a result of illumination by the light sources, and means for applying modulation functions to the emitted light signal to implement code division multiplexing, each light source being associated with a different function, wherein the functions are selected so that there is substantially no cross correlation between the functions, and wherein the light sources have overlapping spectra.

2. A system as claimed in claim 1 comprising demultiplexing means for determining a contribution of each of the light sources to the light signal detected by the light detector.

3. A system as claimed in claim 1, wherein the modulation functions includes using one or more Hadamard or reduced-Hadamard (Simplex) code sequences.

4. A system as claimed in claim 1, wherein the parameter is an oxygenation of the biological tissue.

5. A system as claimed in claim 1, wherein light signals from the biological tissue is one or more of transmitted light; reflected light; backscattered light; fluorescence spectroscopy; or Raman emission.

6. A system as claimed in claim 1, wherein the light signal from the light sources is in an infrared region.

7. A system as claimed in claim 1, wherein each light source has a wavelength bandwidth in a range of 10-140 nm.

8. A system as claimed in claim 7, wherein each light source has a wavelength bandwidth in a the range of 20-100 nm.

9. A system as claimed in claim 1, wherein each light source has a wavelength bandwidth of more than 100 nm.

10. A tool for non-invasive measurement of parameters relating to a biological tissue comprising:
a proximal or handle end that includes a plurality of light sources, each operable to emit a light signal with one or more predetermined wavelengths and a light detector for detecting light signals from the biological tissue as a result of illumination by the light sources; and
a distal end, wherein at least one optical fiber extends between each light source and the distal end for allowing transmission of light from each light source to the distal end and into the biological tissue, and at least one optical fiber extends between the light detector and the distal end to capture light that has passed through the tissue.

11. A tool as claimed in claim 10 that has gripping portions for gripping biological tissue.

12. A tool as claimed in claim 11, wherein the plurality of light sources and the light detector are positioned to direct light into or receive light from one or more gripping faces of the gripping portions.
